(19) United States
(12) Patent Application Publication
(10) Pub. No.: US 2017/0252209 A1
(43) Pub. Date: Sep. 7, 2017

(54) Device, Delivery System, And Method For Ocular Therapy

(71) Applicants: Patrick GOOI, Calgary (CA); Kevin WARRIAN, Calgary (CA)

(72) Inventors: Patrick GOOI, Calgary (CA); Kevin WARRIAN, Calgary (CA)

(21) Appl. No.: 15/503,396

(22) PCT Filed: Aug. 11, 2015
(86) PCT No.: PCT/CA2015/050755

§ 371 Date: Feb. 12, 2017

Related U.S. Application Data
(60) Provisional application No. 62/036,414, filed on Aug. 12, 2014.

(51) Int. Cl. A61F 9/00; A61M 5/28; A61M 5/20; A61F 9/007; A61M 5/19

(52) U.S. Cl. CPC A61F 9/0017; A61F 9/00781; A61M 5/19; A61M 5/2053; A61M 5/284; A61M 5/288; A61M 2005/1787

(57) ABSTRACT

Devices and methods for the ab interno delivery of two or more fluid compositions into Schlemm's canal through a single insertion point in the eye with minimal trauma. The hand-held delivery device includes a handle coupled to an elongated shaft that terminates in a self-trephinating tip through which one or more cannulae are slidably disposed and can be extended or retracted therethrough. The handle further defines a chamber for receiving one or more cartridges each containing at least one fluid composition. Each cartridge is fluidly connected to a respective cannula such that sequential actuation of each cartridge causes dispensation of the fluid compositions at the site for delivery without having to repeatedly enter the target site multiple times. Ab interno methods for localized delivery of a fluid composition into Schlemm's canal are further described.

### PRIORITY

The application claims the benefit of the filing date of U.S. Provisional Application No. 62/036,414, filed Aug. 12, 2014, the disclosure which is herein incorporated by reference in its entirety.

### FIELD OF THE INVENTION

The present invention relates to the field of ocular therapies and, in particular, to a device, a system, and a method for sequential ab interno delivery of two or more fluid compositions through a single insertion point in the eye. Ab interno methods for localizing the delivery of a fluid composition to Schlemm's canal are further described.

### BACKGROUND OF THE INVENTION

Glaucoma is a disease that affects over 60 million people worldwide, or about 1-2% of the population. The disease is typically characterized by an elevation in eye pressure (intraocular pressure) that causes pathological changes in the optic nerve which if left untreated can cause blindness. The increased intraocular pressure is generally caused by a resistance to drainage of aqueous humor or fluid from the eye.

Aqueous humor is a clear, colourless fluid that is continuously replenished by the ciliary body in the eye and then ultimately exits the eye through the trabecular meshwork. The trabecular meshwork extends circumferentially around the eye in the anterior chamber angle and feeds outwardly into a narrow circumferential passageway generally surrounding the exterior border of the trabecular meshwork (Schlemm's canal). From Schlemm's canal, aqueous humor empties into aqueous collector channels or veins positioned around and radially extending from Schlemm's canal. Pressure within the eye is determined by a balance between the production of aqueous humor and its exit through the trabecular meshwork. Resistance to flow in the trabecular meshwork and/or Schlemm's canal can cause decreased flow of aqueous humor out of the eye and increased intraocular pressure.

Treatments that reduce intraocular pressure can slow or stop progressive loss of vision associated with some forms of glaucoma and such treatments are currently the primary therapy for glaucoma. A number of treatment methods are currently used for reducing intraocular pressure to treat glaucoma including medication, laser therapies and various forms of surgery. Drug therapy includes topical ophthalmic drops or oral medications that either reduce the production or increase the outflow of aqueous humor. When medical and laser therapy fail, however, more invasive surgical therapy is typically used.

Surgical techniques for treating glaucoma generally involve the mechanical disruption of the trabecular meshwork. Trabeculectomy, a procedure which is widely practiced, involves microsurgical dissection to mechanically create a new drainage pathway for aqueous humor to drain, by removing a portion of sclera and trabecular meshwork at the drainage angle. Trabeculectomy, however, carries risk of blockage of the surgically-created opening through scarring or other mechanisms and has been found to have limited long-term success. Furthermore, trabeculectomy surgery is associated with serious, potentially blinding complications.

Alternative surgical procedures to trabeculectomy include tube shunt surgeries, non-penetrating trabeculectomy and viscocanalostomy. These procedures are invasive as they are “ab externo” (from the outside of the eye). Tube shunt surgeries involve significant extraocular and intraocular surgery with significant risk of surgical complications, as well as the long term risk of failure from scarring. In the case of viscocanalostomy and non-penetrating trabeculectomy, the procedures involve making a deep incision into the sclera and creating a scleral flap to expose Schlemm's canal for cannulation and dilation. Due to the delicate nature of these ab-externo approaches, they are difficult to execute.

Due to the invasiveness of such procedures and the difficulty of successfully accessing the small diameter of Schlemm's canal from the outside of the eye, “ab interno” techniques have been described for delivering ocular devices and compositions into Schlemm's canal through the trabecular meshwork from the inside of the eye.

International Patent Publication No. WO2013/141898 describes a system and method for accessing and for delivering an ocular device or fluid composition into Schlemm's canal. A device is described that comprises a handle and a cannula configured to facilitate accessing the lumen of Schlemm's canal. A positioning element or conduit is slidable within the cannula to deliver an ocular device or a fluid composition within Schlemm's canal in a minimally traumatic manner in order to mechanically disrupt and dilate Schlemm's canal to improve flow through the trabecular meshwork.

International Patent Publication No. WO2013/158919 describes a delivery system that can be used to deliver an ocular implant into a target location within the eye via an ab interno procedure. The delivery system comprises a handle component and a distal delivery component that includes a guide wire that can be extended distally relative to a sheath to disengage a mounted implant and/or to deliver fluid through openings in the guide wire to mechanically disrupt and/or dilate Schlemm's canal.

While such delivery systems provide means for accessing Schlemm's canal in an ab interno manner in order to mechanically disrupt blockages to aqueous humor flow, there remains a continuing need for alternative approaches to treating and delivering ocular therapies to Schlemm's canal.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present disclosure is to provide a delivery system and method for ocular therapy. In accordance with one aspect, there is described a hand-held delivery device for ab interno delivery of two or more fluid compositions through a single insertion point in the eye, comprising: a handle having a distal end and a proximal end, the distal end coupled to an elongated shaft that terminates in a self-trephinating tip; a chamber defined by the handle and configured to receive one or more fluid-filled cartridges, wherein each fluid-filled cartridge is charged by a respective piston to cause dispensation of the fluid from the respective cartridge; and one or more cannulae slidably disposed within the elongated shaft and having a distal end and a proximal end, wherein the distal end of each of the one or more cannulae is distally extendable from the tip, the proximal end of each of the one or more cannulae is in fluid communication with the chamber and is adapted for fluid connection to the one or more fluid-filled cartridges when inserted in the chamber; wherein actuation of the respective piston allows controllable dispensation of two or more fluids contained in the fluid-filled cartridges for delivery through the fluidly connected cannulae in a sequential manner.

In accordance with another aspect, there is described a hand-held delivery device for ab interno delivery of two or more fluid compositions through a single insertion point in the eye, comprising: a handle having a distal end and a proximal end, the distal end coupled to an elongated shaft that terminates in a self-trephinating tip, the handle defining a chamber configured to receive one or more fluid-filled cartridges; one or more fluid-filled cartridges each comprising a first interior chamber for containing a fluid composition and separated from a second interior chamber by a piston, wherein the second interior chamber is adapted to receive a drive fluid for charging the respective piston to cause dispensation of the fluid composition from the first interior chamber; and one or more cannulae slidably disposed within the elongated shaft and having a distal end and a proximal end, wherein the distal end of each of the one or more cannulae is distally extendable from the tip, the proximal end of each of the one or more cannulae is in fluid communication with the chamber and is adapted for fluid connection to the first interior chamber of the one or more fluid-filled cartridges inserted in the chamber; wherein actuation of the respective piston allows controllable dispensation of two or more fluids contained in the fluid-filled cartridges for delivery through the fluidly connected cannulae in a sequential manner.

In accordance with a further aspect, there is described a system for ab interno delivery of two or more fluid compositions through a single insertion point in the eye, comprising the hand-held delivery device according to embodiments described herein, and a drive fluid source, wherein the two or more fluid compositions are delivered in a sequential manner.

In accordance with another aspect, there is described an ab interno method for sequential delivery of two or more fluid compositions through a single insertion point in the eye, comprising: (a) entering the eye by inserting a self-trephinating tip of a hand-held delivery device, wherein the hand-held delivery device comprises a handle having a distal end and a proximal end, the distal end coupled to an elongated shaft that terminates in the self-trephinating tip, and the handle defining a chamber housing one or more fluid-filled cartridges; (b) extending a first cannula into Schlemm's canal, wherein the first cannula is slidably disposed within the elongated shaft and is distally extendable at a first end through the tip of the hand-held delivery device, and wherein the first cannula is fluidly connected to the one or more fluid-filled cartridges at a second end; (c) actuating the one or more fluid-filled cartridges to dispense a first fluid composition from the one or more fluid-filled cartridges through the fluidly connected first cannula to deliver the first fluid composition into Schlemm's canal; (d) extending a second cannula into Schlemm's canal, wherein the second cannula is slidably disposed within the elongated shaft and is distally extendable at a first end through the tip of the hand-held delivery device, and wherein the second cannula is fluidly connected to the one or more fluid-filled cartridges at a second end; and (e) actuating the one or more fluid-filled cartridges to dispense a second fluid composition from the one or more fluid-filled cartridges through the fluidly connected second cannula to deliver the second fluid composition into Schlemm's canal; wherein steps (b) to (e) are repeated to sequentially deliver additional fluid compositions to the eye.

In accordance with a further aspect, there is described an ab interno method for localized delivery of a fluid composition to Schlemm's canal through a single insertion point in the eye, comprising: (a) entering Schlemm's canal by inserting a self-trephinating tip of a hand-held delivery device, wherein the hand-held delivery device comprises a handle having a distal end and a proximal end, the distal end coupled to an elongated shaft that terminates in the self-trephinating tip, and the handle defining a chamber housing one or more fluid-filled cartridges; (b) extending a first cannula into Schlemm's canal, wherein the first cannula is slidably disposed within the elongated shaft and is distally extendable at a first end through the tip of the hand-held delivery device, and wherein the first cannula is fluidly connected to the one or more fluid-filled cartridges at a second end; (c) actuating the one or more fluid-filled cartridges to dispense a first fluid composition from the one or more fluid-filled cartridges through the fluidly connected first cannula to deliver the first fluid composition into Schlemm's canal; and (d) actuating the one or more fluid-filled cartridges to dispense an ophthalmic viscosurgical device (OVD) from the one or more fluid-filled cartridges through a fluidly connected second cannula, wherein the OVD is delivered at the insertion point in Schlemm's canal to form a plug, wherein the OVD plug maintains localization of the first fluid composition in Schlemm's canal.

### BRIEF DESCRIPTION OF THE DRAWINGS

These and other features of the invention will become more apparent in the following detailed description in which reference is made to the appended drawings.

FIG. 1 is a cross-sectional view of an eye, showing the location of the trabecular meshwork and Schlemm's canal;

FIGS. 2A and 2B are perspective views of a hand-held delivery device for delivering multiple fluid compositions to the eye, according to embodiments of the present disclosure, in an assembled position (FIG. 2A) and in a disassembled position (FIG. 2B);

FIG. 3 is a cross-sectional view of the hand-held delivery device shown in FIG. 2;

FIGS. 4A and 4B illustrate ab interno accessibility to the full circumference of Schlemm's canal by the hand-held delivery device for delivering multiple fluid compositions to the eye;

FIG. 5 is a cross-sectional view of the handle of the hand-held delivery device with piston actuated cartridges inserted;

FIGS. 6A to 6D are various views of a hand-held delivery device having a rotary magazine cartridge system for delivering multiple fluid compositions through a single access point; FIG. 6A is a schematic view of a hand held delivery device illustrating lining up of the cannula with each cartridge; FIG. 6B is a perspective view of a hand-held delivery device without the cartridges installed in the rotary magazine; FIG. 6C is a perspective view of a hand-held delivery device with the rotary cartridges installed; FIG. 6D is a cross-sectional view of the hand-held delivery device shown in FIG. 6B;

FIGS. 7A and 7B are cross-sectional views of a serially arranged cartridge system for delivering multiple fluid compositions through a single access point using the hand-held delivery device;

FIG. 8A to 8C are a schematic diagram of a drive fluid system for ab interno delivery of two or more fluid compositions, and a drive fluid source;

FIG. 9 is a schematic diagram showing the extendable cannula tethered to an advancing panel, which is in contact with the roller wheel mechanism;

FIG. 10 is a schematic diagram showing hands free extension of the cannula;

FIG. 11 is a perspective view of a shaft tip for both high viscosity and low viscosity fluid delivery of the hand-held delivery device; and

FIG. 12 One variation of the embodiment is that that there is no extendable cannula, but the lumens from chamber 1 and 2 empty into the common hollow tapered tip of the hand-held delivery device.

### DETAILED DESCRIPTION OF THE INVENTION

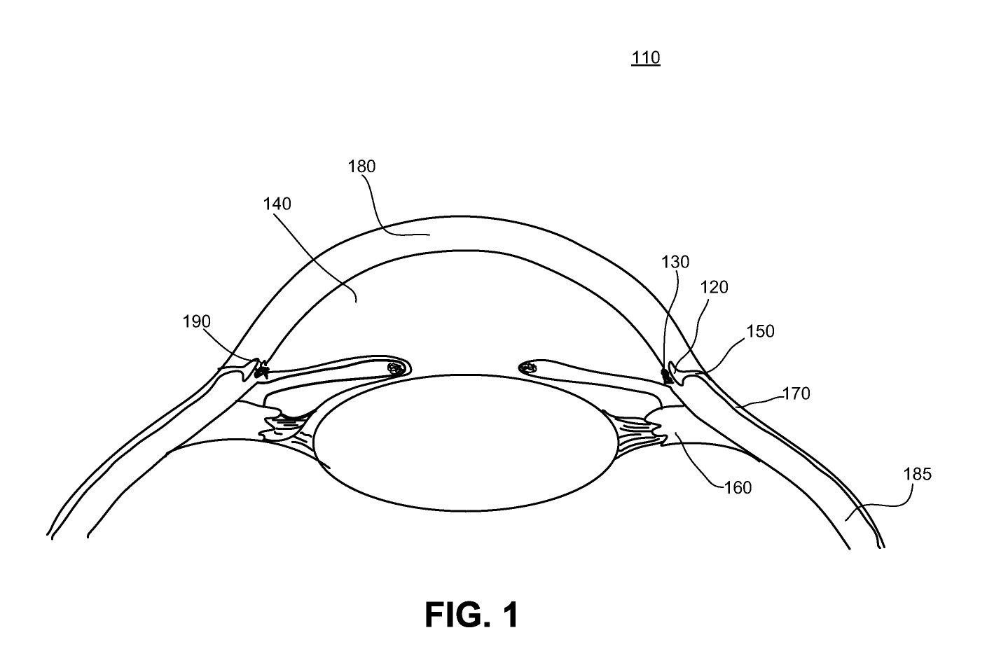

Referring to FIG. 1, a cross-section of an eye 110 is illustrated to show the relative anatomy of Schlemm's canal 120, the trabecular meshwork 130, and the anterior chamber 140. The anterior chamber 140 is bound anteriorly by the cornea 180 which is connected on its periphery to the sclera 185, a tough fibrous tissue forming the white shell of the eye 110. The trabecular meshwork 130 is located on the outer periphery of the anterior chamber 140 and extends 360° circumferentially around the anterior chamber 140 with Schlemm's canal 120 also extending 360° circumferentially around the outer peripheral surface of the trabecular meshwork 130.

The anterior chamber 140 of the eye 110 is filled with aqueous humor which is produced by the ciliary body 160 to ultimately exit the eye 110 through the trabecular meshwork 130. In a normal eye 110, aqueous humor passes through the trabecular meshwork 130 into Schlemm's canal 120 and thereafter through a plurality of aqueous veins 170, which merge with blood-carrying veins (not shown), and into systemic venous circulation. Intraocular pressure is maintained by balancing this secretion and outflow of aqueous humor. As discussed, glaucoma is characterized by an excessive buildup of aqueous humor, which leads to an increase in intraocular pressure that is distributed relatively uniformly throughout the eye 110.

Accessing Schlemm's canal 120 for treating glaucoma has primarily focused on means for mechanically disrupting the trabecular meshwork 130 and juxtacanalicular tissue (not shown) in order to maintain the good communication to Schlemm's canal 120. Described in the present disclosure are devices and methods that offer alternative approaches for delivering therapeutic treatment to the eye 110. According to embodiments described, devices and methods are disclosed for the ab interno delivery of two or more fluid compositions through a single insertion point 190 in the eye 110 with minimal trauma. In particular, devices and methods for delivering two or more fluid compositions into Schlemm's canal 120 are described such that Schlemm's canal 120 can be treated in a 360° manner with minimal trauma.

Unless defined otherwise, all technical and scientific terms used herein have the same meaning as commonly understood by one of ordinary skill in the art to which this invention belongs.

As used herein, the terms “medicament”, “therapeutic”, “drug”, “bioactive compound”, and “bioactive agent” are used interchangeably to refer to one or more ingredients having bioactive, medicinal, or prophylactic properties.

As used herein, the term “fluid composition” refers to a composition in a liquid or gas form. According to embodiments of the present disclosure, the composition may or may not include one or more medicament, therapeutic, drug, bioactive compound, and/or bioactive agent. For example, fluid compositions according to embodiments of the present disclosure can include without limitation saline, ophthalmic viscosurgical devices (OVDs), air, and/or in combination with one or more medicament, therapeutic, drug, bioactive compound, and/or bioactive agent with potential to also inject a compound designed to counteract other bioactive agents previously administered.

As used herein, the term “disposable” describes articles that are not intended to be restored or reused and which are intended to be discarded after a single use.

As used herein, the term “about” refers to an approximately +/−10% variation from a given value. It is to be understood that such a variation is always included in any given value provided herein, whether or not it is specifically referred to.

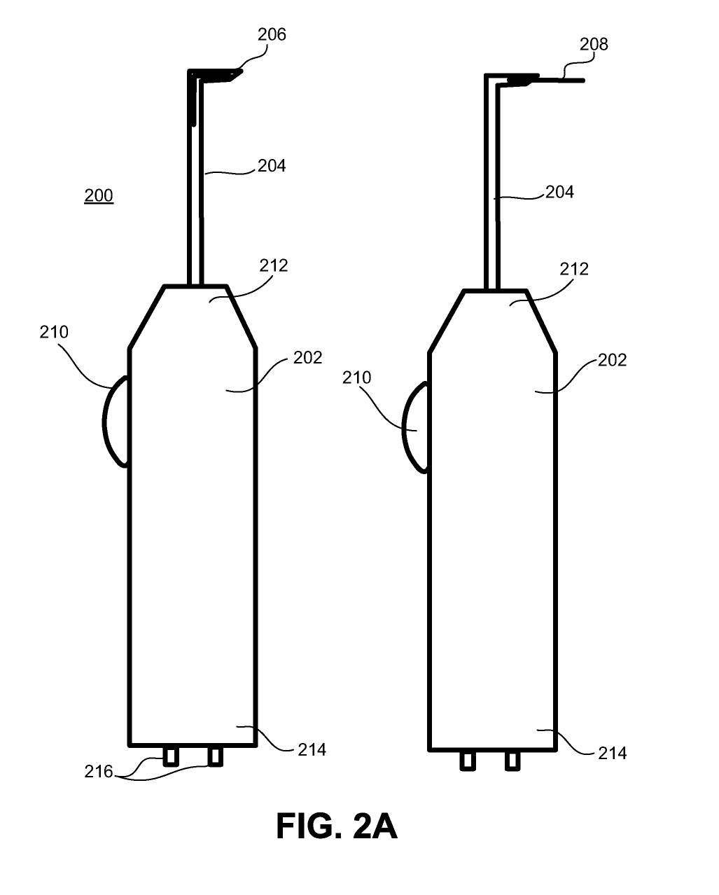

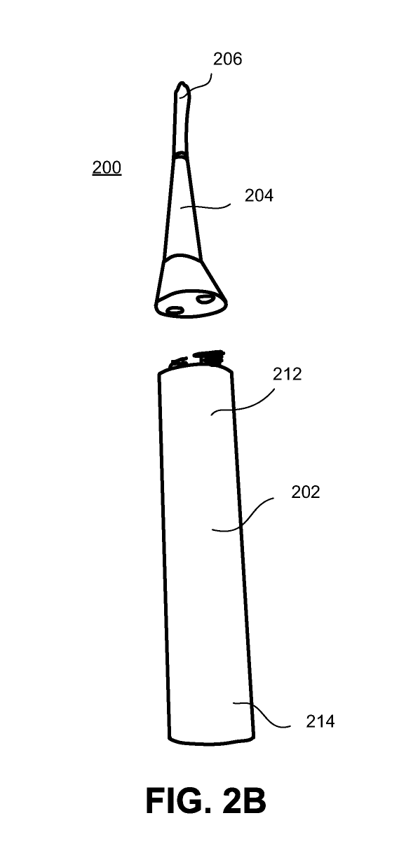

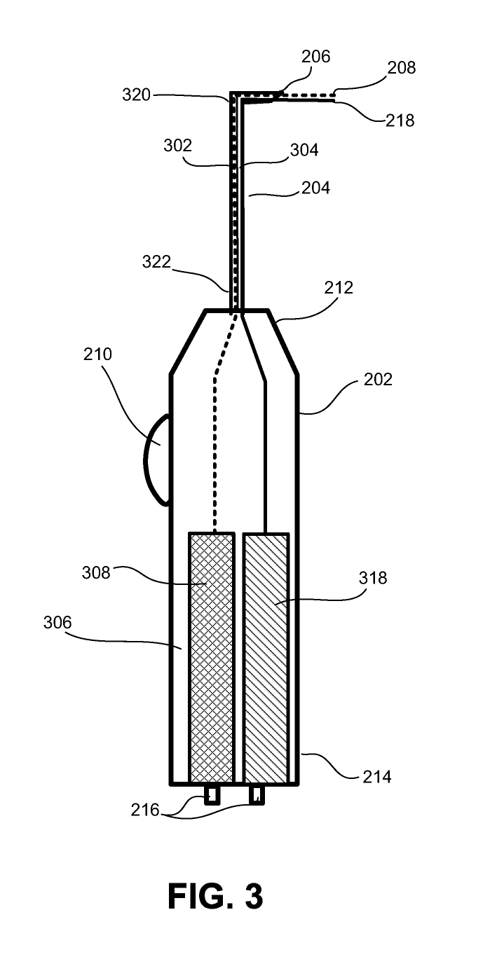

Turning to FIGS. 2A, 2B and FIG. 3, specifically described is a hand-held delivery device 200 is shown in an assembled configuration (FIG. 2A) and in its disassembled form (FIG. 2B) that includes a handle 202 that can be easily manipulated by one hand. The delivery device 200 includes a handle 202 having a distal end 212 and a proximal end 214 that defines an interior chamber 306. The handle 202 is designed for single-handed use in either the right or left hand of the operator. According to certain embodiments, the device can be reversible by rotating the handle 202 180-degrees, thereby accommodating use by both left and right handed surgeons and allowing different portions of the trabecular meshwork 130 to be targeted for entry.

The distal end 212 of the handle 202 is coupled to an elongated shaft 204 that terminates in a self-trephinating tip 206 through which one or more cannulae 208 are slidably disposed and can be extended or retracted therethrough. The self-trephinating tip 206 can take any form known to those skilled in the art. As shown in FIGS. 2A and 2B, for example, the self-trephinating tip 206 can be formed by angling the tip of the shaft 204 to create a sharp point 206 that can facilitate piercing of the trabecular meshwork 130 and smooth entry into Schlemm's canal 120. According to certain embodiments, the self-trephinating tip 206 can be curved up to about 90° relative to the axis of the shaft 204 in order to further facilitate entry into Schlemm's canal 120 with minimal trauma and to create a seal with the inner wall 150 once inserted. The fluid-filled cartridges 308, 318 are fluidly coupled to their respective cannulae 208, 218 and at the other end are fluidly coupled to a Luer-lock connector 216 secured to IV tubing 806. Pressure from the fluid received via the Luer-lock connector 216 causes the fluid in the cartridges 308, 318 to flow out the tip 206.

As shown in FIG. 3, the shaft 204 defines at least one lumen, in this example there are two lumen 302, 304 to allow one or more cannulae 208, 218 to be slidably disposed within the elongated shaft 204. The shaft 204 is sized, therefore, to accommodate the slidably disposed cannulae 208, 218 while being small enough to minimize trauma to Schlemm's canal 120. The outer diameter of the shaft 204 can range from about 150 microns to about 1600 microns. The inner diameter can range from about 50 microns to about 1200 microns. The shaft 204 can be made from any suitable material having sufficient stiffness to allow it to be advanced through the eye wall and anterior chamber. For example, metals such as stainless steel, nickel, titanium, aluminum, or alloys thereof, or a polymer, can be used. Exemplary polymers include without limitation, polycarbonate, polyetheretherketone, polyethylene, polypropylene, polyimide, polyamide, polysulfone, polyether block amide, and fluoropolymers.

The one or more cannulae 208, 218, slidably disposed within the lumen 302, 304 of the elongated shaft 204, each have a distal end 320 that is distally extendable from the tip 206. The proximal end 322 of each of the one or more cannulae 208, 218 is in fluid communication with the chamber 306 and is adapted for fluid connection to one or more fluid-filled cartridges 308, 318 that are insertable within the chamber 306. The cannulae 208, 218 are sized to slidably fit within the lumen 302, 304 of the shaft 204 and sized to be able to be extended through Schlemm's canal 120 in order to deliver one or more fluid compositions in a targeted manner. The outer diameter of the cannulae 208, 218 can range from about 50 microns to about 500 microns, from about 300 microns to about 500 microns, from about 200 microns to about 250 microns, or from about 180 microns to about 300 microns.

The cannulae 208, 218 can be made from any suitable material that imparts the desired combination of flexibility and rigidity for introduction through the eye wall 180 accessing Schlemm's canal 120, and/or navigation through other ocular tissue structures. For example, the cannulae 208, 218 can comprise a polymer; a polymer reinforced with metal wire, braid or coil; composites of polymers and metal; or metals such as stainless steel, titanium, nitinol, or alloys thereof. The cannulae 208, 218 can be straight with enough flexibility and rigidity to be forcibly advanced through Schlemm's canal 120.

The cannulae 208, 218 are slidably disposed within the shaft lumen 302, 304 so as to be extendable and retractable through the tip 206 of the shaft 204. An ab interno method is described for localized delivery of a fluid composition into Schlemm's canal 120 through a single insertion point 190 in the eye 110. Schlemm's canal 120 is entered by inserting the tip 206 of the device 200. The cannulae 208, 218 are extendable into Schlemm's canal 120 by any suitable amount and direction about the circumference 402 of the canal 120 to deliver a first fluid composition into Schlemm's canal 120. Once the first fluid composition is delivered as desired, the cannula 208 is retracted and an OVD is delivered through the second cannula 218 at the insertion point 190 in Schlemm's canal 120 to form a plug. In this way, the OVD plug prevents reflux of the first fluid composition and maintains localization of the first fluid composition in Schlemm's canal 120. The second cannula 218 can be extended in order to deliver the OVD plug in order to avoid interference with the first cannula 208. Alternatively, the second cannula 218 can deliver the OVD plug from a retracted position in the shaft lumen 204. Such methods may be used to treat glaucoma, pre-glaucoma, ocular hypertension, or other ocular conditions wherein treatment through Schlemm's canal 120 may be advantageous.

The methods of the present disclosure generally involve making an incision 420 in the ocular wall 180 to provide access to the anterior chamber 140 of the eye 110. Such methods are well known to those skilled in the art and will not be discussed in detail here. Once the incision 420 is made, the self-trephinating tip 206 of the hand-held delivery device 200 can be inserted through the trabecular meshwork 130 to access Schlemm's canal 120. A first cannula 208 can then be extended through the lumen of the shaft 204 into Schlemm's canal 120. The cannula 208 can be extended into Schlemm's canal 120 by any suitable amount and direction depending on the particular procedure being conducted.

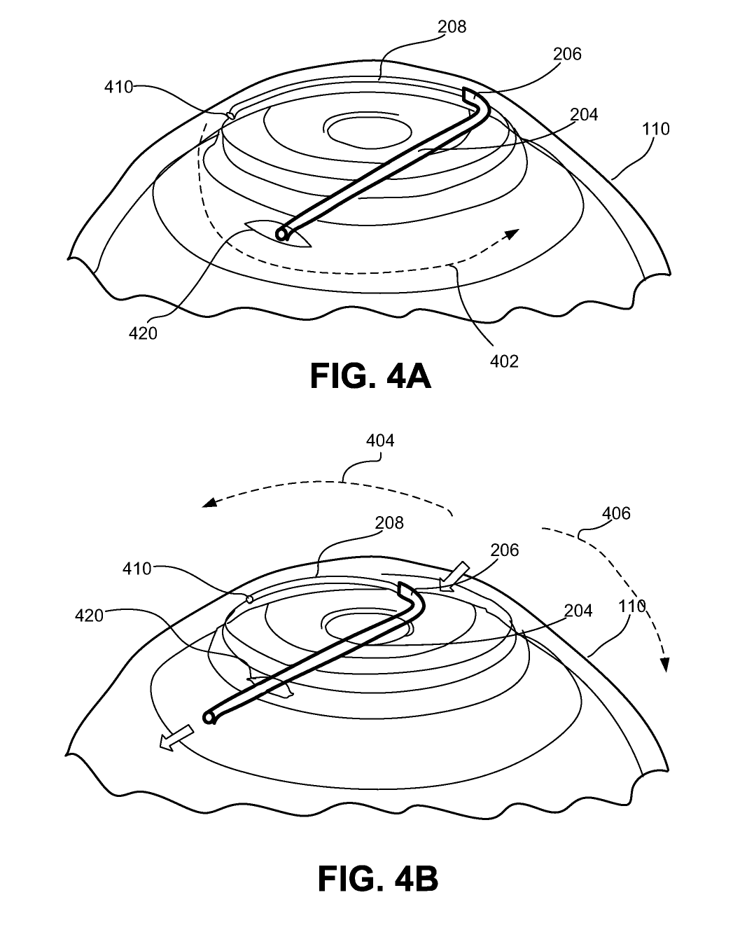

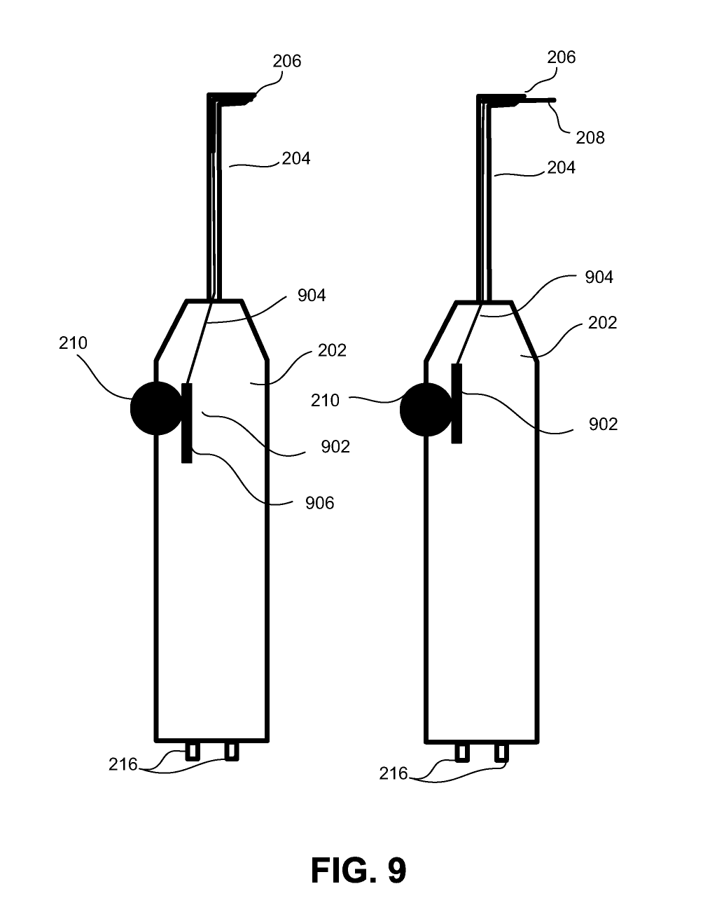

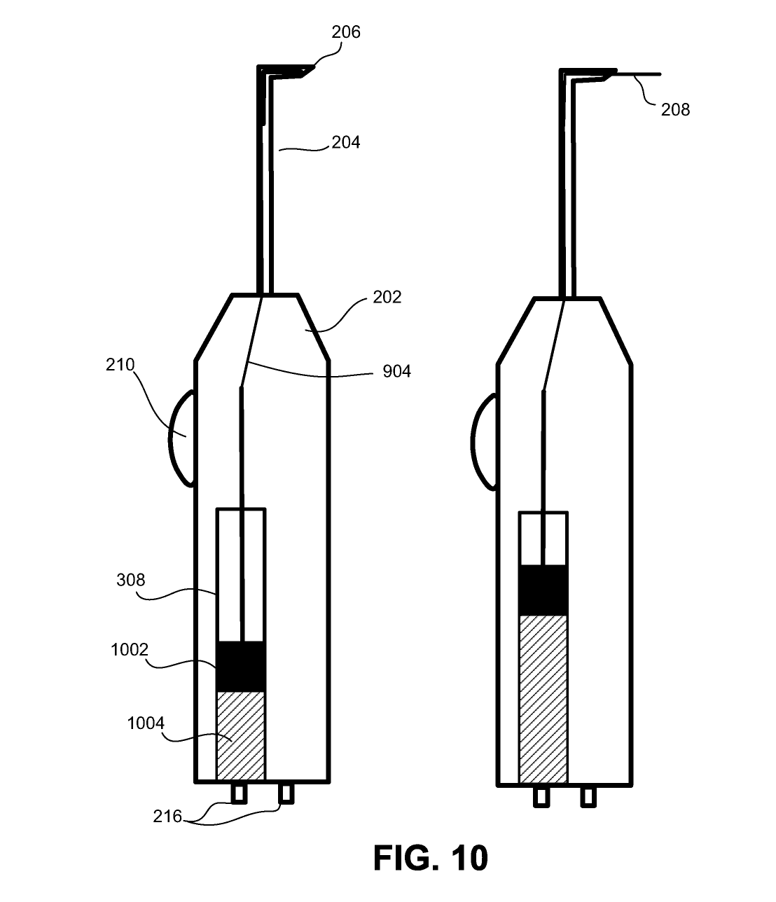

In this way, as shown in FIGS. 4A and 4B, for example, the cannulae 208, 218 can be extended incrementally around the partial circumference, the entire circumference 402, or to any desired extent around the circumference 402 of Schlemm's canal 120, in order to deliver a fluid composition through the distal end 410 of the cannula 208, and therefore provide treatment to, the desired extent of the canal 120 and up to the entire circumference 402 of the canal 120. According to embodiments of the present disclosure, the cannulae 208, 218 are extendable between about 10° to about 360° about the canal. According to other embodiments as shown in FIG. 4B, the cannulae 208, 218 can be extended through Schlemm's canal 120 in multiple steps, e.g., 180° in a counter-clockwise direction 404 and 180° in a clockwise direction 406 about the canal 120, in order to achieve delivery of a fluid composition, and therefore treatment, to the full 360° circumference 402 of Schlemm's canal 120. In other embodiments, the cannula 208 can be extended through Schlemm's canal 120 in a 90° arc. It will be readily apparent to those skilled in the art that the amount and direction of the extension of the cannula 208 into Schlemm's canal 120, through the single access point, can be varied in any number of combinations to achieve the desired level of access into Schlemm's canal 120 without repeated exit and re-entry of the cannula 208 from the canal 120. Fluid can be injected during or upon extension or retraction of the cannula 208 through the distal end of the cannula 208. Extension and/or retraction of the cannulae 208, 218 is controllable by an operably connected controller mechanism 210 located on the exterior of the handle 202 so as to be easily accessible to the operator with one hand. The drive mechanism is described with reference to FIGS. 9 and 10 below.

As previously mentioned, the handle 202 further defines an interior chamber 306 that is sized to receive one or more cartridges 308, 318 each containing at least one fluid composition. Each cartridge 308, 318 is fluidly connected to a respective cannula 208, 218 such that sequential actuation of each cartridge 308, 318, during extension of the cannula 208, 218 about Schlemm's canal 120 for example, causes dispensation of the fluid compositions at the site for delivery without having to repeatedly enter the insertion point 190, e.g., Schlemm's canal 120, multiple times. In this way, multiple treatment fluid compositions such as saline, ophthalmic viscosurgical devices (OVD), medicaments, and bioactive agents, can be separately delivered to the target site in a sequential manner through a single insertion point 190, thereby minimizing trauma to the eye 110.

Using the hand-held delivery device 200 disclosed herein the method generally includes entering Schlemm's canal 120 and actuating the corresponding fluid-filled cartridge 308, 318 to sequentially dispense one or more fluid compositions through the fluidly connected cannula(e) 208, 218. The fluid composition(s) are localized in Schlemm's canal 120 by dispensing an ophthalmic viscosurgical device (OVD) (not shown) from the one or more fluid-filled cartridges 308, 318 through a fluidly connected cannula 208 to form a plug at the insertion point 190 in Schlemm's canal 120. In this way, the OVD plug limits the fluid composition(s) delivered into Schlemm's canal 120 from refluxing out of the canal 120 and thereby maintains localization of the delivered fluid composition(s) in Schlemm's canal 120. Localization of the delivered fluid composition(s), according to certain embodiments, can further be ensured by delivering a further OVD plug while withdrawing the hand-held delivery device 200 from Schlemm's canal 120 in order to maintain compartmentalization of the anterior chamber 140 of the eye 110. The ability to localize therapeutics, for example, within Schlemm's canal 120, presents alternative approaches to ocular therapy while minimizing physical or biochemical trauma to the eye 110.

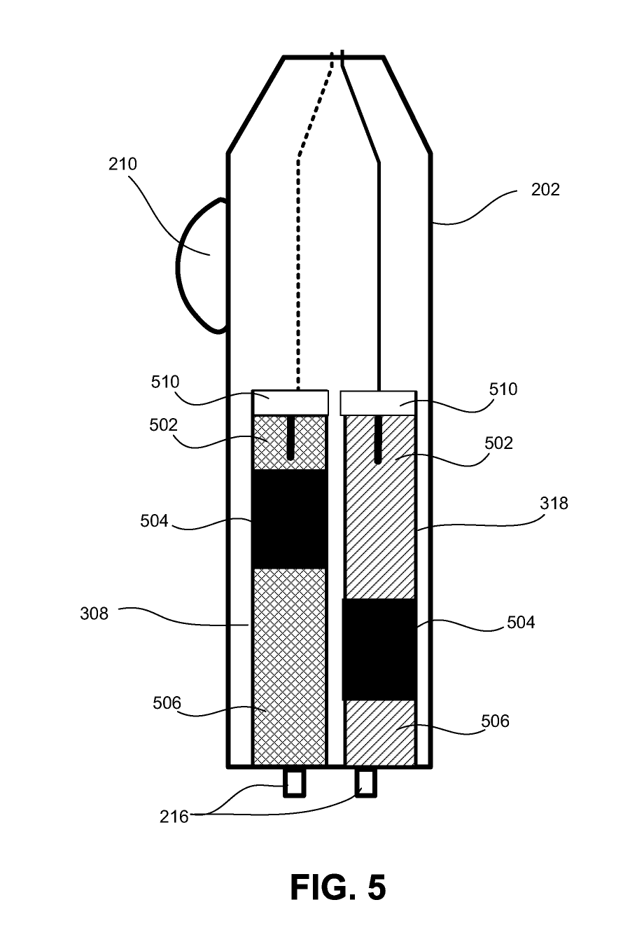

An alternative fluid-filled cartridge 308, 318 is shown in FIG. 5 the hand-held delivery device 200 can be adapted to comprise two fluid-filled cartridges 308, 318. According to such embodiments, each cartridge 308, 318 contains a different fluid composition and is in fluid connection with a respective cannula 208, 218 for sequential delivery into Schlemm's canal 120 through a single insertion point 190. Each cartridge 308, 318 is adapted for fluid connection of a first interior chamber 502 with a respective cannula 208, 218. For example, the cartridge 308, 318 can present screw-type threads 510 at the first interior chamber 502 end that matingly engage with corresponding threads presented at the end of each respective cannula 208, 218. Cartridges 308, 318 can, in this way, be replaced as needed. According to certain embodiments, the cartridges 308, 318 are interchangeable and can be replaced between uses in the hand-held delivery device 200.

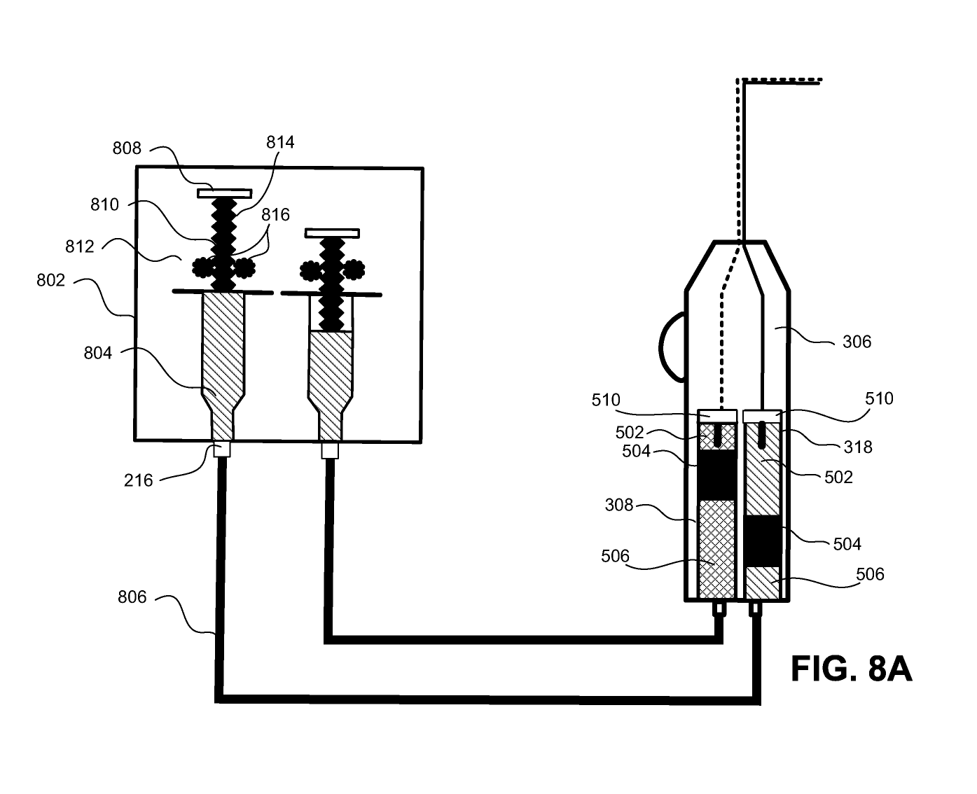

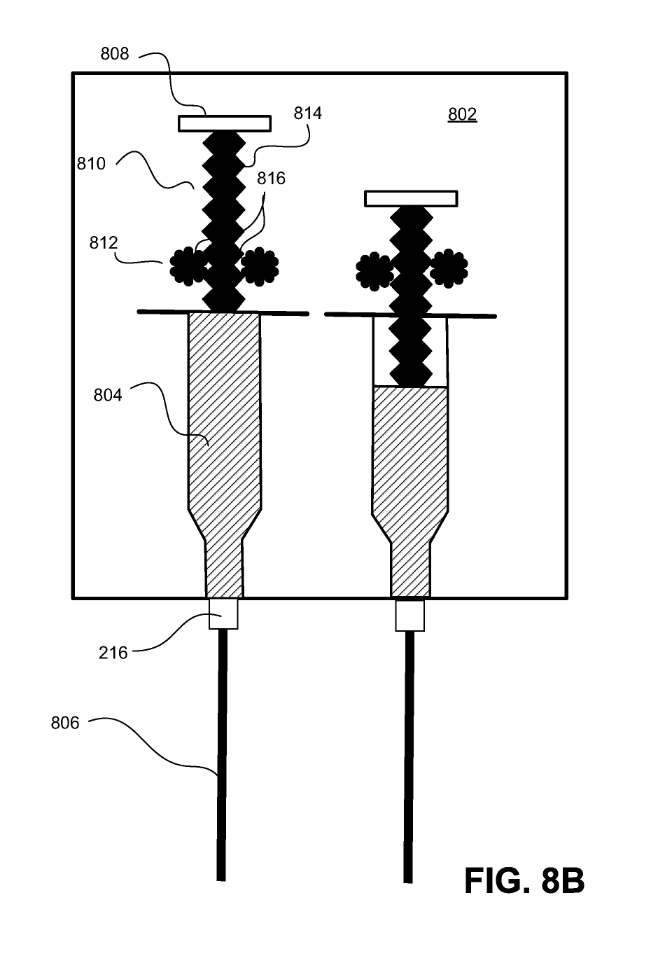

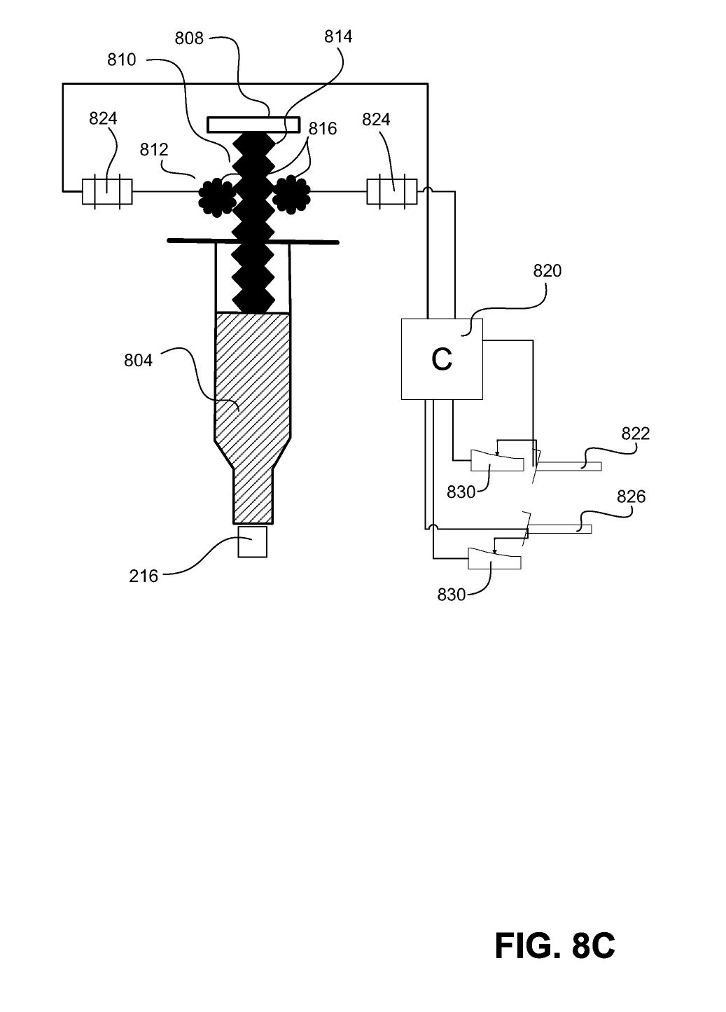

The first interior 502 chamber for containing a fluid composition is separated from a second interior 506 chamber by a piston or plunger 504. The second interior chamber 506 of each cartridge 308, 318 is fluidly connected, for example by a drive fluid conduit (not shown), to a drive fluid source 802, shown in more detail with reference to FIG. 8A to 8C below, which when activated fills the second interior chamber 506 with drive fluid, charging the piston 504 to drive into the first interior chamber 502 and, thereby, displacing the fluid composition for delivery. The dispensation of the fluid composition from the first interior chamber 502 is through fluidly connected cannula(e) 208, 218 that can be extended into Schlemm's canal 120 for targeted delivery. The drive fluid source, according to embodiments, can be fluidly connected to the fluid-filled cartridge 308, 318, for example, with a Luer-lock connector 216 to secure the drive fluid conduit 806 to the fluid-filled cartridge 308, 318. The drive fluid source 802, which is further described with reference to FIGS. 8A and 8B below, can be motorized and controlled by a foot pedal 822. In this way, the system of the present disclosure can be operated with one hand and by a single unassisted operator.

Sequential actuation of each respective piston 504 allows controllable dispensation of each fluid composition from the fluid-filled cartridges 308, 318 such that each fluid composition is alternately delivered into Schlemm's canal 120 in a sequential manner. According to certain embodiments, controlled volumes of liquid can be delivered under low-flow conditions. For the intentions of ophthalmology, the volumes of fluids can range from about 50 μL-2000 μL.

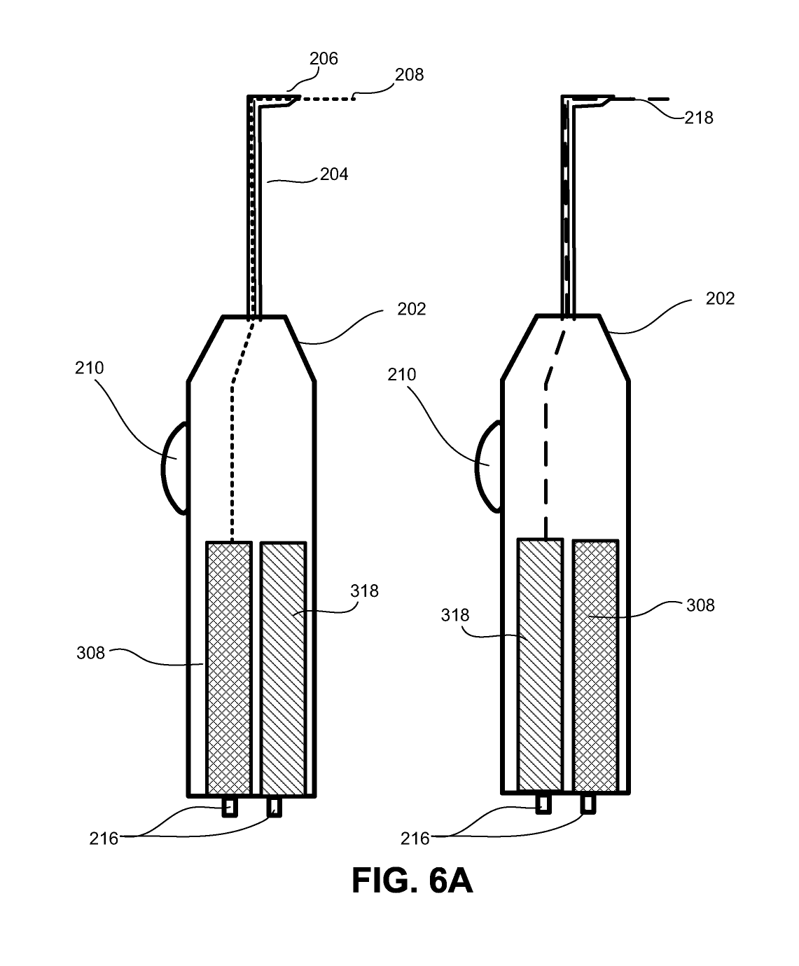

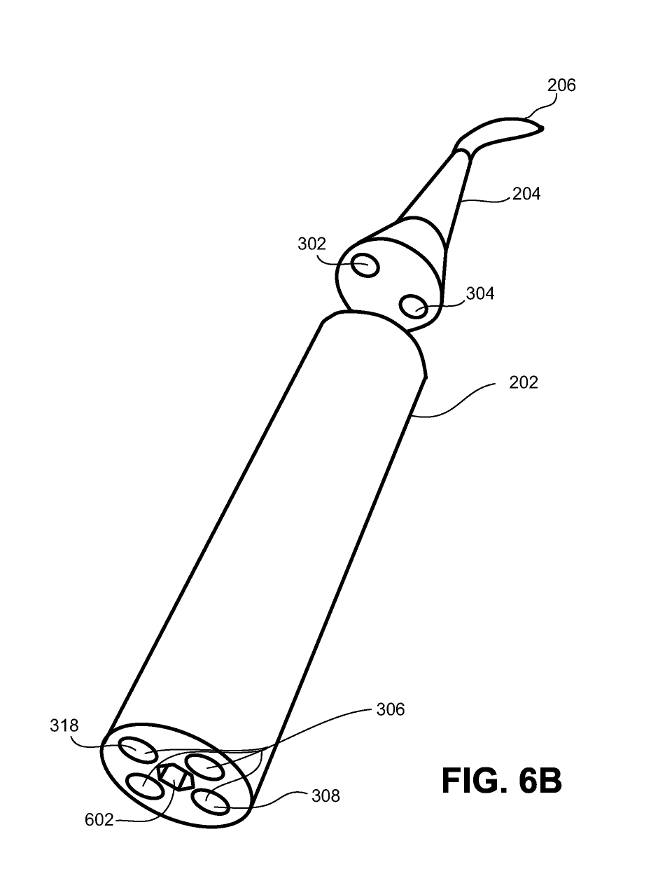

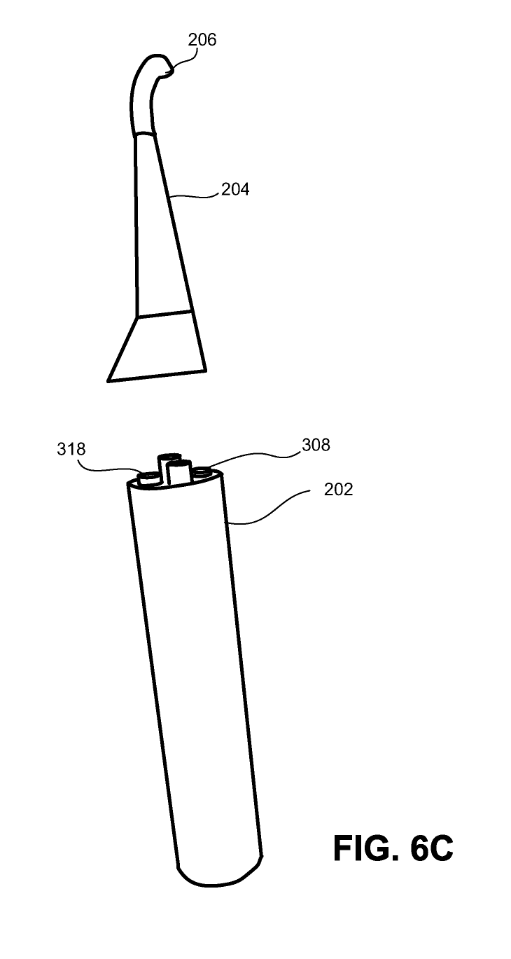

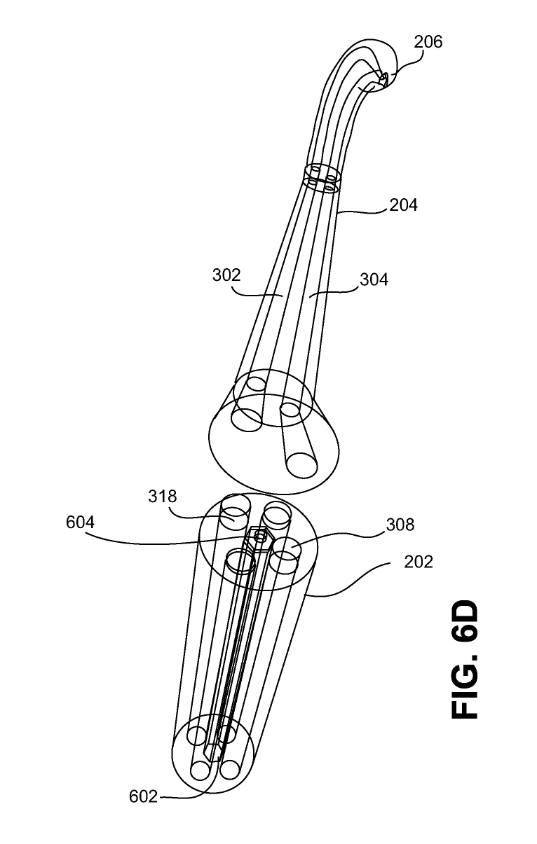

Alternatively or in addition to the other embodiments described herein, the hand-held device 200 can be adapted to have chambers 306 to receive multiple cartridges 308, 318 assembled on a rotating carousel 602 shown in FIGS. 6A to 6D. Rotation of the carousel 602 allows sequential alignment and fluid connection of each fluid-filled cartridge 308, 318 with the cannula 208, 218 for delivery through the incision 420 in the eye 110. Once delivery of the first fluid composition has been achieved, the first cannula 208 can be retracted from Schlemm's canal 120 while continuing to maintain the position of the tip 206 in the canal. A second cannula 218, fluidly connected to a second fluid-filled cartridge 318, can then be extended into Schlemm's canal 120 for controlled delivery of a second fluid composition into the canal 120 in a similar way as described above. The rotating carousel 602 may have two chambers 306 to receive cartridges 308, 318 and a single cannula 204 with flows 208 and 218 flowing through the single cannula 204. Alternatively, the rotating carousel 602 may have four chambers 306 for receiving up to four cartridges 308, 318 and have two cannulae 302, 304. In some embodiments, each fluid-filled cartridge 308, 318 contains a different fluid composition. The rotary magazine may be rotated manually or by a motorized device located centrally 604 within the handle 202 so that a different cartridge 308, 318 lines up with the lumen 302, 304 of the shaft 204.

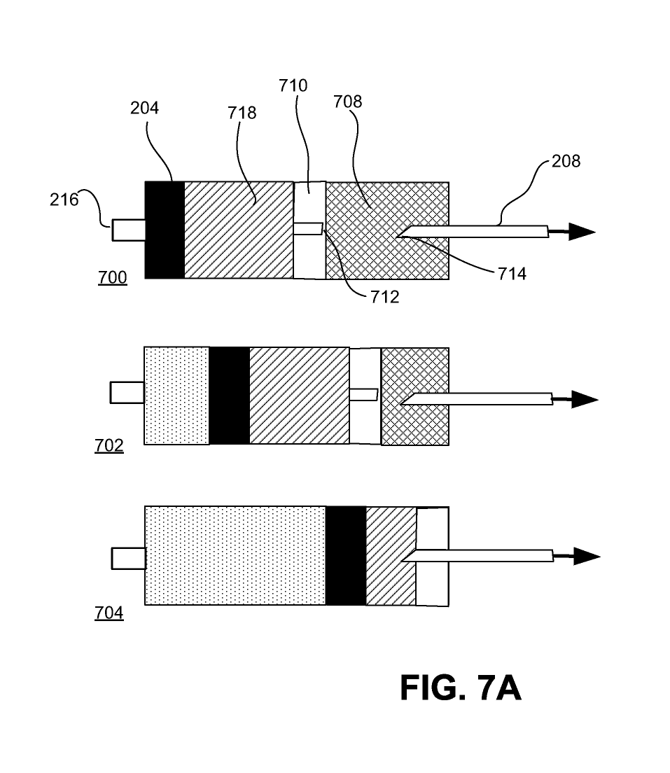

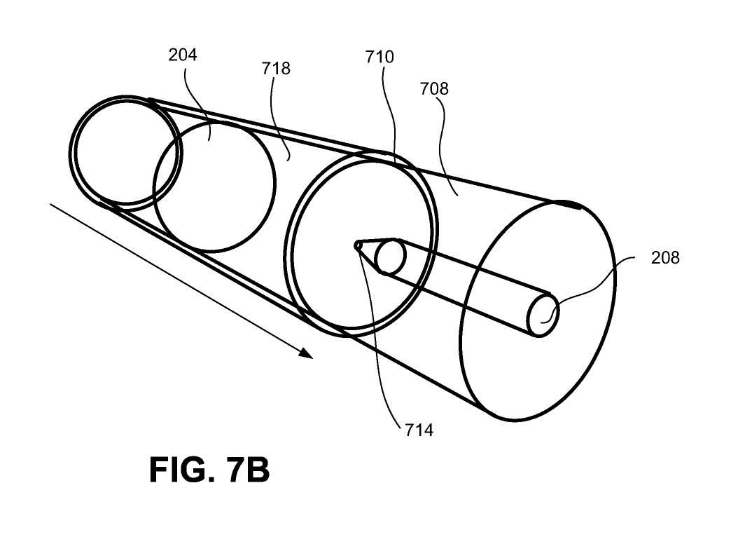

Alternatively or in addition to the other embodiments described herein, the cartridge 308, 318 can comprise multiple discrete compartments 708, 718 each separately containing a different fluid composition as shown in FIGS. 7A and 7B. The two compartments 708, 718 are separated by a plunger 710 having a weakened or narrowed membrane 712 located generally in the center of the plunger 710. As fluid is injected 702 from the Luer-lock connector 216, the piston 204 advances displacing fluid from the first discrete compartment 708 into the cannula 208 via a sharpened tip 714. When all the fluid in the first compartment 708 has been emptied, the sharpened tip 714 punctures the membrane 712 of the plunger 710 permitting fluid from the second chamber 718 to flow out of the cannula 208. By having multiple plungers 710 with membranes 712 and a suitably long sharpened tip 714, more than one fluid composition may be loaded into a single cartridge position for sequential delivery into Schlemm's canal 120. The amount of fluid in each compartment 708, 718 may vary depending on the particular procedure.

Alternatively or in addition to the other embodiments described herein, the hand-held delivery device 200 can be controllably activated by any drive fluid source 802 adapted for charging the fluidly connected piston 504 of the one or more cartridges 308, 318 to allow controllable dispensation of the fluids contained in each cartridge 308, 318 as shown in FIGS. 8A and 8B. Specifically, each fluid-filled cartridge 308, 318 is fluidly connected to a drive fluid source 802 which when activated fills the second interior chamber 506 of the cartridge 308, 318 with drive fluid from a syringe 804 to controllably charge the piston or plunger 504 to drive into the first interior chamber 502 thereby displacing the fluid composition for delivery.

The drive fluid source 802 may take the form of a motorized injection and aspiration system comprising at least one syringe 804 fluidly coupled to a drive fluid conduit 806 and having a plunger 808 with a shaft 810 for forcibly advancing a drive fluid through the drive fluid conduit 806. The drive fluid conduit 806 is configured, at the end opposite the syringe 804, for fluid connection to the hand-held delivery device 200. The drive fluid conduit 806 fluidly connects the drive fluid-filled syringe 804 to the second interior chamber 506 of a cartridge 308, 318 inserted in a chamber 306 of the hand-held device 200. In this way, expulsion of the drive fluid from the syringe 804 into the second interior chamber 506 charges the respective piston 504 to cause the fluid composition contained in the first interior chamber 502 to be displaced and dispensed through the fluidly connected cannula 208, 218 to the target site in Schlemm's canal 120. According to certain embodiments, the syringe 804 can include a Luer-Lok type connector 216 for fluid coupling to the drive fluid conduit 806.

Drive fluid is driven from the syringe 804 by actuation of a syringe driver 812. According to some embodiments, the drive fluid is a balanced salt solution (BSS); however, any fluid may be used to drive the syringe driver 812. The syringe driver 812, according to some embodiments, comprises a series of cogs 814 that line the exterior of the plunger shaft 810. The series of cogs 814 cooperate with a gear system 816 that is operatively connected to an axle (not shown). In such embodiments, the movement of the gears 816 along the cogs 814 allows for incremental advancement of the plunger 808 translating into the injection pressure at the outlet of the fluidly connected cannula 208, 218.

In operation, axle rotation is controlled through a control unit 820 in communication with one or more step motors 824, the syringe driver 812, and at least one foot pedal 822. According to such embodiments, actuation of the foot pedal 822 by the operator allows controllable movement of the plunger 808 between a raised and lowered position within the syringe 804. In this way, the drive fluid source 802 is adapted for hands-free or single hand control.

The step motor 824 is controllable by actuation of a foot pedal 822. For example, depressing the foot pedal 822 causes a signal to be sent to the controller 820 which in turn activates the one or more step motors 824. The step motors 824 rotate the gears 816 to advance the plunger 808 within the syringe 804. Toggling the foot pedal 822, for example by kicking the pedal 822 to the side, may send a different signal to be sent to the controller 820 which the controller 820 causes the step motors 824 to reverse direction. The reverse rotation causes gears 816 to retract the plunger 808 from the syringe 804 thereby causing aspiration to occur.

Alternatively, or in addition to the other embodiments, the step motor 824 can be controllable by a dual foot pedal 822, 826. For example, depression of one foot pedal 822 can cause advancement of the plunger 808 and depression of the other foot pedal 826 can cause retraction of the plunger 808. The degree of depression of the dual foot pedals 822, 826 similarly can govern the speed of the advancement and retraction.

Alternatively, or in addition to the other embodiments, the degree of depression or toggling of the foot pedal 822 dictates to the controller 820 the speed of advancement and the degree of toggling dictates to the controller 820 the speed of the retraction from the syringe 804. The foot pedal 822, 826 can include potentiometers 830 sending signals to the controller 820 that function as accelerators to increase the speed of motors 824 thereby increasing the plunger 808 advancement or retraction as the degree of depression of the pedal 822, 826 increases from a resting position. A control unit 820 may further control the maximum speed of the step motor 824 to ensure safe operation. Such control systems are familiar to those skilled in the art and can be operatively connected to the foot pedal 822, 826 and/or step motor 824 through electrical hardwiring or wireless communication.

According to some embodiments, referring to the embodiments illustrated in FIG. 9, the cannulae 208, 218 are tethered to a roller-wheel driven mechanism 902 that is located on the exterior of the handle 202 and easily operational by the operator by scrolling or sliding the roller wheel 210. The extendable cannula 208 is tethered to an advancing panel 906, which is in contact with the roller wheel 210. Therefore, as the roller-wheel 210 is turned, the advancing panel 906 changes position, and the cannula 208 may be extended or retracted from the tip 206. An alternative embodiment is for a sliding mechanism instead of a roller-wheel mechanism 902. In this embodiment, the sliding mechanism is connected to the advancing panel 906, which either extends or retracts the cannula 208. This can be a manual mechanism, or a mechanism driven by an electric or pneumatic motor (not shown). The latter mechanism may be more feasible given the delicate nature of the procedure.

According to some embodiments, the device 200 can be adapted for hands-free control of the cannulae 208, 218 as shown with reference to FIG. 10. For example, extension and retraction of the cannulae 208, 218 can be under foot control. The cannula 208 may be tethered 904 to a plunger 1002 within cartridge 308, which is connected to a foot controlled drive fluid system as described in FIGS. 8A to 8C, or alternatively for example the Low Rider System. When fluid is injected into chamber 1004 by Luer-lok 216, the displacement of the plunger 1002 causes the tether 904 to extend. Thereby, foot control of the exemplary Low Rider System can be used to control the advancement of the plunger 1002, which in turn advances or retracts the extendable cannula 208.

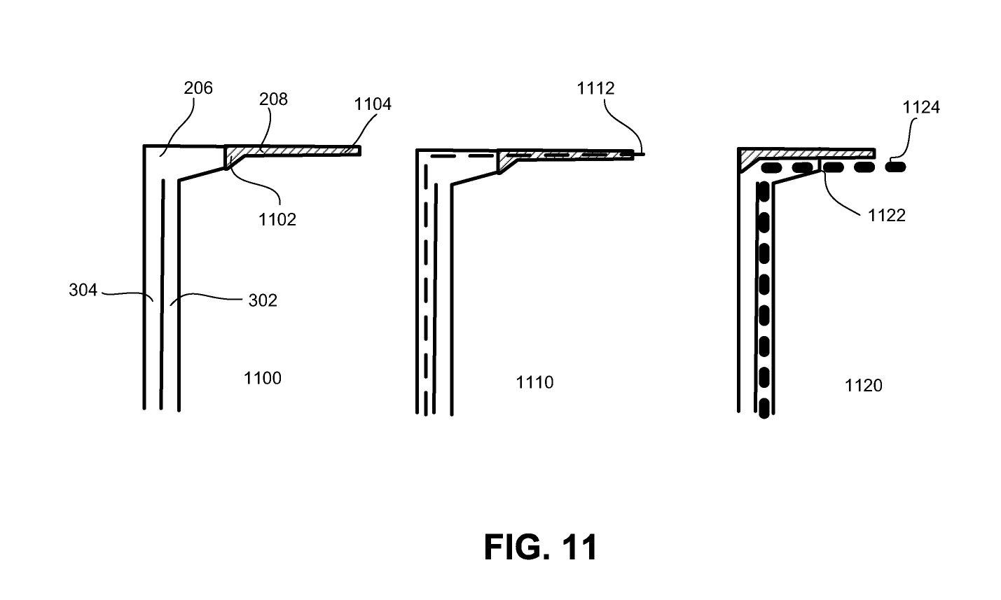

Alternatively or in addition to the other embodiments described herein, an alternative tip 206 for delivering high viscosity and low viscosity fluids into the Schlemm's canal 120 is shown in FIG. 11. When the cannula 208 is fully extended 1100, the flared base 1102 of the cannula only permits fluid from the lumens 302, 304 to exit via the narrowed opening 1104. The narrowed gauge opening 1104 is intended for less viscous fluid 1112 to flow from cartridge 308 via lumen 304 and is able to flow out of the extendable cannula 208. When the cannula 208 is fully retracted 1120, the opening 1122 is larger than the opening 1104 of the cannula 208. The larger opening 1122 permits more viscous fluid 1124 to flow freely, without the risk of clogging, from the tip 206. Or alternatively, a higher flow rate of a less viscous fluid 1112 may flow from the larger opening 1122.

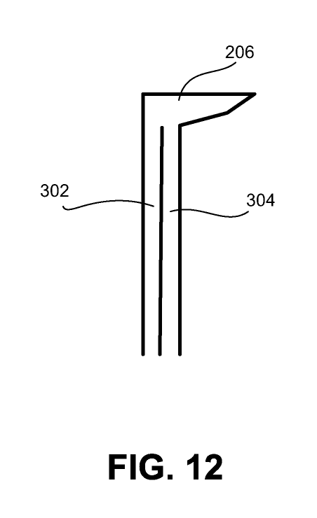

In an alternative embodiment, the tip 206 may not have an extendable cannula 208, 218 as shown in FIG. 12. The fluid flowing from the lumens 302, 304 flows from a widened tip 206. The tip, as mentioned before is self trephinating and designed with possibly a tapered edge so as to limit or reduce reflux of the therapeutic out of Schlemm's canal into the rest of the anterior chamber. Since the tip partially blocks the goniotomy leading to the Schlemm's canal, this limits or reduces the volume of therapeutic reaching unintentional targets.

According to certain embodiments, the hand-held delivery device 200 can be adapted to include viewing means, such as a fiber optic or one or more compound lenses in order to facilitate insertion and extension of the device 200 in the eye.

The fluid compositions that may be delivered by the hand-held device 200 described herein includes medicaments, saline and ophthalmic viscosurgical device (OVD), for example. The OVDs may comprise hyaluronic acid, chondroitin sulfate, cellulose, derivatives or mixtures thereof, or solutions thereof. In one variation, the OVDs are dispersive-type OVDs. Examples of medicaments that may be delivered using the methods described herein may include a drug suitable for treating glaucoma, reducing or lowering intraocular pressure, reducing inflammation or scarring, and/or preventing infection. According to further embodiments, the methods of the present disclosure may be used to deliver bioactive agents having certain bioactive properties that may be desirable for ocular treatment through Schlemm's canal 120. According to further embodiments, the fluid compositions may also include agents that aid with visualization of the fluid composition. For example, dyes such as but not limited to fluorescein, trypan blue, or indocyanine green may be included. In some variations, a fluorescent compound or bioluminescent compound is included in the fluid composition to help with its visualization. It is also contemplated that air or a gas could be delivered by the described methods.

According to certain embodiments, a further volume of OVD may be delivered while withdrawing the hand-held delivery device 200 from Schlemm's canal 120 in order to form an additional plug to maintain compartmentalization of the anterior chamber 140 of the eye 110.

According to certain embodiments, the hand-held delivery device 200 is disposable. According to other embodiments, the hand-held delivery device 200 can be re-used and is amenable to known sterilization techniques for reuse.

Although the embodiments described herein demonstrate ab interno procedures, the inventor contemplates that the embodiments may also be used for certain ab externo procedures.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

The embodiments of the invention in which an exclusive property or privilege is claimed are defined as follows:

1. A hand-held delivery device for delivery of two or more fluid compositions through a single insertion point in the eye, comprising:
a handle having a distal end and a proximal end, the distal end coupled to an elongated shaft that terminates in a self-trephinating tip;
a chamber defined by the handle and configured to receive one or more fluid-filled cartridges, wherein each fluid-filled cartridge is charged by a respective piston to cause dispensation of the fluid from the respective cartridge; and
one or more cannulae slidably disposed within the elongated shaft and having a distal end and a proximal end, wherein the distal end of each of the one or more cannulae is distally extendable from the tip, the proximal end of each of the one or more cannulae is in fluid communication with the chamber and is adapted for fluid connection to the one or more fluid-filled cartridges when inserted in the chamber;
wherein actuation of the respective piston allows controllable dispensation of two or more fluids contained in the fluid-filled cartridges for delivery through the fluidly connected cannulae in a sequential manner.

2. The hand-held delivery device according to claim 1, wherein the tip is angled relative to the shaft to facilitate insertion.

3. The hand-held delivery device according to claim 1, wherein the handle comprises a wheeled drive assembly operably connected to the one or more cannulae for controllably extending or retracting the one or more cannulae from the tip.

4. The hand-held delivery device according to claim 1, wherein the chamber is configured to receive two fluid-filled cartridges, wherein each fluid-filled cartridge contains a different fluid composition, and wherein each fluid-filled cartridge is respectively in fluid connection with the one or more cannulae for sequential delivery through the single insertion point in the eye.

5. The hand-held delivery device according to claim 1, wherein the chamber is configured to receive one fluid-filled cartridge, wherein the fluid-filled cartridge comprises two or more discrete compartments each containing a different fluid composition, and wherein the fluid-filled cartridge is in fluid connection with the cannula for delivery through the single insertion point in the eye.

6. The hand-held delivery device according to claim 1, wherein the chamber is configured to receive a rotating carousel having multiple fluid-filled cartridges held therein, wherein each fluid-filled cartridge contains a different fluid composition, and wherein rotation of the carousel allows sequential alignment and fluid connection of each fluid-filled cartridge with the one or more cannulae for delivery through the single insertion point in the eye.

7. The hand-held delivery device according to claim 1, wherein at least one of the two or more fluid compositions comprises a medicament, a therapeutic, a bioactive agent, or a mixture thereof.

8. The hand-held delivery device according to claim 1, wherein at least one of the two or more fluid compositions comprises an ophthalmic viscosurgical device (OVD).

9. The hand-held delivery device according to claim 8, wherein the OVD comprises hyaluronic acid, chondroitin sulfate, cellulose, or salts, derivatives, or mixtures thereof.

10. The hand-held delivery device according to claim 8, wherein the two or more fluid compositions further comprises a medicament, a therapeutic, a bioactive agent, or a mixture thereof.

11. The hand-held delivery device according to claim 1, wherein each fluid-filled cartridge comprises a first interior chamber for containing the fluid composition, the first interior chamber adapted for fluid connection to the respective cannula and separated from a second interior chamber by the piston, wherein the second interior chamber is adapted to receive a drive fluid for charging the respective piston to cause dispensation of the fluid composition from the first interior chamber and through the cannula.

12. The hand-held delivery device according to claim 11, wherein the second interior chamber of each fluid-filled cartridge is respectively connected to a drive fluid source.

13. The hand-held delivery device according to claim 12, wherein the drive fluid source is motorized.

14. The hand-held delivery device according to claim 13, wherein the drive fluid source is foot-controlled.

15. The hand-held delivery device according to claim 1, further comprising a viewing means to aid in determining placement of the device.

16. The hand-held delivery device according to claim 15, wherein the viewing means is a fiber optic or a compound lens.

17. A hand-held delivery device for ab interno delivery of two or more fluid compositions through a single insertion point in the eye, comprising:
a handle having a distal end and a proximal end, the distal end coupled to an elongated shaft that terminates in a self-trephinating tip, the handle defining a chamber configured to receive one or more fluid-filled cartridges;
one or more fluid-filled cartridges each comprising a first interior chamber for containing a fluid composition and separated from a second interior chamber by a piston, wherein the second interior chamber is adapted to receive a drive fluid for charging the respective piston to cause dispensation of the fluid composition from the first interior chamber; and
one or more cannulae slidably disposed within the elongated shaft and having a distal end and a proximal end, wherein the distal end of each of the one or more cannulae is distally extendable from the tip, the proximal end of each of the one or more cannulae is in fluid communication with the chamber and is adapted for fluid connection to the first interior chamber of the one or more fluid-filled cartridges inserted in the chamber;
wherein actuation of the respective piston allows controllable dispensation of two or more fluids contained in the fluid-filled cartridges for delivery through the fluidly connected cannulae in a sequential manner.

18. The hand-held delivery device according to claim 17, wherein the second interior chamber of each fluid-filled cartridge is respectively connected to a drive fluid source.

19. The hand-held delivery device according to claim 18, wherein the drive fluid source is motorized.

20. The hand-held delivery device according to claim 19, wherein the drive fluid source is foot-controlled.

21. The hand-held delivery device according to claim 17, comprising two fluid-filled cartridges, wherein each fluid-filled cartridge contains a different fluid composition, and wherein each fluid-filled cartridge is in fluid connection with a respective cannula for sequential delivery through the single insertion point in the eye.

22. The hand-held delivery device according to claim 17, comprising one fluid-filled cartridge, wherein the fluid-filled cartridge comprises two or more discrete compartments each containing a different fluid composition, and wherein the fluid-filled cartridge is in fluid connection with the cannula for delivery through the single insertion point in the eye.

23. The hand-held delivery device according to claim 17, comprising multiple fluid-filled cartridges on a rotating carousel, wherein each fluid-filled cartridge contains a different fluid composition, and wherein rotation of the carousel allows sequential alignment and fluid connection of each fluid-filled cartridge with the cannula for delivery through the single insertion point in the eye.

24. The hand-held delivery device according to claim 17, wherein at least one of the two or more fluid compositions comprises a medicament, a therapeutic, a bioactive agent, or a mixture thereof.

25. The hand-held delivery device according to claim 17, wherein at least one of the two or more fluid compositions is an ophthalmic viscosurgical device (OVD).

26. The hand-held delivery device according to claim 25, wherein the OVD comprises hyaluronic acid, chondroitin sulfate, cellulose, or salts, derivatives, or mixtures thereof.

27. The hand-held delivery device according to claim 25, wherein the two or more fluid compositions further comprises a medicament, a therapeutic, a bioactive agent, or a mixture thereof.

28. A system for ab interno delivery of two or more fluid compositions through a single insertion point in the eye, comprising the hand-held delivery device according to claim 17 and a drive fluid source, wherein the two or more fluids are delivered in a sequential manner.

29. The system according to claim 28, wherein the drive fluid source is motorized.

30. The system according to claim 29, wherein the drive fluid source is foot-controlled.

31. The system according to claim 28, wherein the drive fluid source comprises:
at least one syringe fluidly coupled to a drive fluid conduit and having a plunger with a shaft for forcibly advancing a drive fluid through the drive fluid conduit, wherein the drive fluid conduit at an end opposite the syringe is configured for fluid connection to the hand-held delivery device;
a syringe driver for driving the plunger;
at least one foot pedal;
a step motor; and
a control unit in communication with the step motor, the syringe driver and the at least one foot pedal.

32. The system according to claim 31, wherein the syringe driver comprises a series of cogs lining the exterior of the shaft, the series of cogs cooperating with a pair of gears operatively connected to an axle, wherein operation of the syringe driver allows the plunger to be controllably raised or lowered within the syringe.

33. The system according to claim 31, wherein the at least one syringe comprises a Luer-Lok type connector configured for fluidly coupling the at least one syringe to the drive fluid conduit.

34. The system according to claim 31, wherein the drive fluid is a balanced salt solution (BSS).

35. The system according to claim 31, comprising two syringes wherein each syringe comprises a drive fluid conduit for fluid connection with a respective fluid-filled cartridge inserted within the hand-held delivery device.

36. The system according to claim 28, comprising two fluid-filled cartridges, wherein each fluid-filled cartridge contains a different fluid composition, and wherein each fluid-filled cartridge is in fluid connection with a respective cannula for sequential delivery through the single insertion point in the eye.

37. The system according to claim 28, comprising one fluid-filled cartridge, wherein the fluid-filled cartridge comprises two or more discrete compartments each containing a different fluid composition, and wherein the fluid-filled cartridge is in fluid connection with the cannula for delivery through the single insertion point in the eye.

38. The system according to claim 28, comprising multiple fluid-filled cartridges on a rotating carousel, wherein each fluid-filled cartridge contains a different fluid composition, and wherein rotation of the carousel allows sequential alignment and fluid connection of each fluid-filled cartridge with the cannula for delivery through the single insertion point in the eye.

39. The system according to claim 28, wherein at least one of the two or more fluid compositions comprises a medicament, a therapeutic, a bioactive agent, or a mixture thereof.

40. The system according to claim 28, wherein at least one of the two or more fluid compositions is an ophthalmic viscosurgical device (OVD).

41. The system according to claim 40, wherein the OVD comprises hyaluronic acid, chondroitin sulfate, cellulose, or salts, derivatives, or mixtures thereof.

42. The system according to claim 40, wherein the two or more fluid compositions further comprises a medicament, a therapeutic, a bioactive agent, or a mixture thereof.

43. An ab interno method for sequential delivery of two or more fluid compositions through a single insertion point in the eye, comprising:
(a) entering the eye by inserting a self-trephinating tip of a hand-held delivery device, wherein the hand-held delivery device comprises a handle having a distal end and a proximal end, the distal end coupled to an elongated shaft that terminates in the self-trephinating tip, and the handle defining a chamber housing one or more fluid-filled cartridges;
(b) extending a first cannula into Schlemm's canal, wherein the first cannula is slidably disposed within the elongated shaft and is distally extendable at a first end through the tip of the hand-held delivery device, and wherein the first cannula is fluidly connected to the one or more fluid-filled cartridges at a second end;
(c) actuating the one or more fluid-filled cartridges to dispense a first fluid composition from the one or more fluid-filled cartridges through the fluidly connected first cannula to deliver the first fluid composition into Schlemm's canal;
(d) extending a second cannula into Schlemm's canal, wherein the second cannula is slidably disposed within the elongated shaft and is distally extendable at a first end through the tip of the hand-held delivery device, and wherein the second cannula is fluidly connected to the one or more fluid-filled cartridges at a second end; and
(e) actuating the one or more fluid-filled cartridges to dispense a second fluid composition from the one or more fluid-filled cartridges through the fluidly connected second cannula to deliver the second fluid composition into Schlemm's canal.

44. The method according to claim 43, wherein the chamber houses two fluid-filled cartridges each containing a different fluid composition, and wherein each fluid-filled cartridge is respectively in fluid connection with a respective cannula for sequential delivery through the single insertion point in the eye.

45. The method according to claim 43, wherein the chamber houses one fluid-filled cartridge, the fluid-filled cartridge comprising two or more discrete compartments each containing a different fluid composition, and wherein the fluid-filled cartridge is in fluid connection with the cannula for delivery through the single insertion point in the eye.

46. The method according to claim 43, wherein the chamber houses a rotating carousel having multiple fluid-filled cartridges held therein, wherein each fluid-filled cartridge contains a different fluid composition, and wherein rotation of the carousel allows sequential alignment and fluid connection of each fluid-filled cartridge with the one or more cannulae for delivery through the single insertion point in the eye.

47. The method according to claim 45 or claim 46, wherein steps (b) to (e) are repeated to sequentially deliver more than two fluid compositions.

48. The method according to claim 43, wherein at least one of the two or more fluid compositions comprises a medicament, a therapeutic, a bioactive agent, or a mixture thereof.

49. The method according to claim 43, wherein at least one of the two or more fluid compositions is an ophthalmic viscosurgical device (OVD).

50. The method according to claim 49, wherein the OVD comprises hyaluronic acid, chondroitin sulfate, cellulose, or salts, derivatives, or mixtures thereof.

51. The method according to claim 49, wherein the two or more fluid compositions further comprises a medicament, a therapeutic, a bioactive agent, or a mixture thereof.

52. The method according to claim 43, wherein each fluid-filled cartridge comprises a first interior chamber for containing the fluid composition, the first interior chamber adapted for fluid connection to the respective cannula and separated from a second interior chamber by a piston, wherein the second interior chamber is adapted to receive a drive fluid for charging the respective piston to cause dispensation of the fluid composition from the first interior chamber and through the cannula.

53. The method according to claim 52, wherein the second interior chamber of each fluid-filled cartridge is respectively connected to a drive fluid source.

54. The method according to claim 53, wherein the drive fluid source is motorized.

55. The method according to claim 53, wherein the drive fluid source is foot-controlled.

56. The method according to claim 53, wherein actuating the one or more fluid-filled cartridges comprises:
actuating a foot pedal to activate a first syringe driver to forcibly advance a first plunger inserted within a first syringe, the first syringe pre-filled with the drive fluid and fluidly coupled to the second interior chamber of a respective first cartridge to cause dispensation of the fluid composition from the first interior chamber of the first cartridge; and
activating the foot pedal to actuate a second syringe driver to forcibly advance a second plunger inserted within a second syringe, the second syringe pre-filled with the drive fluid and fluidly coupled to the second interior chamber of a respective second cartridge to cause dispensation of the fluid composition from the first interior chamber of the second cartridge.

57. The method according to claim 56, wherein the drive fluid is a balanced salt solution (BSS).

58. The method according to claim 43, wherein the two or more fluid compositions is delivered about at least a 90° arc of Schlemm's canal.

59. The method according to claim 43, wherein the two or more fluid compositions is delivered about at least a 180° arc of Schlemm's canal.

60. The method according to claim 43, wherein the two or more fluid compositions is delivered about at least a 360° arc of Schlemm's canal.

61. An ab interno method for localized delivery of a fluid composition to Schlemm's canal through a single insertion point in the eye, comprising:
(a) entering Schlemm's canal by inserting a self-trephinating tip of a hand-held delivery device, wherein the hand-held delivery device comprises a handle having a distal end and a proximal end, the distal end coupled to an elongated shaft that terminates in the self-trephinating tip, and the handle defining a chamber housing one or more fluid-filled cartridges;
(b) extending a first cannula into Schlemm's canal, wherein the first cannula is slidably disposed within the elongated shaft and is distally extendable at a first end through the tip of the hand-held delivery device, and wherein the first cannula is fluidly connected to the one or more fluid-filled cartridges at a second end;
(c) actuating the one or more fluid-filled cartridges to dispense a first fluid composition from the one or more fluid-filled cartridges through the fluidly connected first cannula to deliver the first fluid composition into Schlemm's canal; and
(d) actuating the one or more fluid-filled cartridges to dispense an ophthalmic viscosurgical device (OVD) from the one or more fluid-filled cartridges through a fluidly connected second cannula, wherein the OVD is delivered at the insertion point in Schlemm's canal to form a plug, wherein the OVD plug maintains localization of the first fluid composition in Schlemm's canal.

62. The method according to claim 59, further comprising:
(e) delivering a further volume of OVD while withdrawing the hand-held delivery device from Schlemm's canal, wherein the OVD forms a further plug to maintain the anterior chamber of the eye.

63. The method according to claim 61 or claim 62, wherein the OVD is a dispersive OVD.

64. The method according to claim 63, wherein the dispersive OVD comprises hyaluronic acid, chondroitin sulfate, cellulose, or salts, derivatives, or mixtures thereof.

65. The method according to claim 61, wherein the first fluid composition comprises a medicament, a therapeutic, a bioactive agent, or a mixture thereof.

66. The method according to claim 61, further comprising: actuating the one or more fluid-filled cartridges to sequentially dispense additional fluid compositions from the one or more fluid-filled cartridges through the fluidly connected cannulae prior to step (d).

67. The method according to claim 61, wherein the chamber houses two fluid-filled cartridges, a first fluid-filled cartridge containing the first fluid composition and a second fluid-filled cartridge containing the OVD, wherein each fluid-filled cartridge is respectively in fluid connection with a respective cannula for sequential delivery through the single insertion point in the eye.

68. The method according to claim 66, wherein the chamber houses one fluid-filled cartridge, the fluid-filled cartridge comprising two or more discrete compartments each containing a different fluid composition at least one of which containing the OVD, and wherein the fluid-filled cartridge is in fluid connection with the cannula for delivery through the single insertion point in the eye.

69. The method according to claim 66, wherein the chamber houses a rotating carousel having multiple fluid-filled cartridges held therein, wherein each fluid-filled cartridge contains a different fluid composition at least one of which containing the OVD, and wherein rotation of the carousel allows sequential alignment and fluid connection of each fluid-filled cartridge with the one or more cannulae for delivery through the single insertion point in the eye.

70. The method according to claim 61, wherein each fluid-filled cartridge comprises a first interior chamber for containing the fluid composition, the first interior chamber adapted for fluid connection to the respective cannula and separated from a second interior chamber by a piston, wherein the second interior chamber is adapted to receive a drive fluid for charging the respective piston to cause dispensation of the fluid composition from the first interior chamber and through the cannula.

71. The method according to claim 70, wherein the second interior chamber of each fluid-filled cartridge is respectively connected to a drive fluid source.

72. The method according to claim 71, wherein the drive fluid source is motorized.

73. The method according to claim 71, wherein the drive fluid source is foot-controlled.

74. The method according to claim 71, wherein actuating the one or more fluid-filled cartridges comprises:
actuating a foot pedal to activate a first syringe driver to forcibly advance a first plunger inserted within a first syringe, the first syringe pre-filled with the drive fluid and fluidly coupled to the second interior chamber of a respective first cartridge to cause dispensation of the fluid composition from the first interior chamber of the first cartridge; and
activating the foot pedal to actuate a second syringe driver to forcibly advance a second plunger inserted within a second syringe, the second syringe pre-filled with the drive fluid and fluidly coupled to the second interior chamber of a respective second cartridge to cause dispensation of the fluid composition from the first interior chamber of the second cartridge.

75. The method according to claim 74, wherein the drive fluid is a balanced salt solution (BSS).

76. The method according to claim 61, wherein the fluid composition is delivered about at least a 90° arc of Schlemm's canal.

77. The method according to claim 61, wherein the fluid composition is delivered about at least a 180° arc of Schlemm's canal.

78. The method according to claim 61, wherein the fluid composition is delivered about at least a 360° arc of Schlemm's canal.

79. A hand-held delivery system for delivery of two or more fluid compositions, comprising:
a shaft having a tip at one end and a handle at the other with at least one lumen therein configured to convey the fluid compositions from a plurality of fluid-filled cartridges to the tip;
at least one piston within each fluid-filled cartridge separating the fluid composition from a drive fluid; and
a drive fluid source supplying the drive fluid, via at least one drive fluid conduit, to the fluid-filled cartridge to cause the at least one piston to displace the fluid composition from the fluid-filled cartridge into the at least one lumen and out the tip.

80. A fluid-filled cartridge comprising:
a first interior chamber configured to be in fluid communication with a cannula;
a piston separating the first interior chamber from a second interior chamber; and
the second interior chamber configured to be in fluid communication with a drive fluid source.

81. The fluid-filled cartridge according to claim 80 further comprising a plunger between the piston and the first interior chamber defining a third interior chamber.

82. The fluid-filled cartridge according to claim 81 wherein the plunger comprises a narrowed membrane configured to be broken by a tip of the cannula.
